(19) United States
(12) Patent Application Publication
(10) Pub. No.: US 2026/0007344 A1
(43) Pub. Date: Jan. 8, 2026

(54) INDIVIDUALIZED PREDICTION OF ACUTE MEDICAL CONDITION INDICATION ONSET USING ACTIMETRIC SENSOR DATA

(71) Applicant: Centre for Addiction and Mental Health, Toronto (CA)

(72) Inventors: Tania Abigail Ortiz Dominguez, Toronto (CA); Ramzi El Halabi, North York (CA)

(21) Appl. No.: 19/259,991

(22) Filed: Jul. 3, 2025

Related U.S. Application Data
(60) Provisional application No. 63/668,759, filed on Jul. 8, 2024.
(60) Provisional application No. 63/668,702, filed on Jul. 8, 2024.

(51) Int. Cl. A61B 5/16; A61B 5/00

(52) U.S. Cl. CPC A61B 5/165; A61B 5/4809; A61B 5/681; A61B 5/7239; A61B 5/7253; A61B 5/7264; A61B 5/7275; A61B 5/7282; A61B 5/746

(57) ABSTRACT

A sensor-based system, method and computer program product for individualized prediction of onset of a symptom of a psychiatric illness or medical condition. Actimetric patient data is obtained from at least one actimetric sensor associated with a patient. The actimetric patient data comprises at least one of time series sleep variable data and time series activity variable data. Multi-resolution time-frequency analysis is applied to the actimetric patient data to obtain time-frequency spike train data for the patient, and onset of a symptom is predicted from the time-frequency spike train data. An alert is issued when the onset of the symptom is predicted from the time-frequency spike train data.

This invention was made with government support under R21MH123849 awarded by the National Institutes of Health. The government has certain rights in the invention.

### TECHNICAL FIELD

The present disclosure relates to transformation of actimetric sensor data to enable prediction of symptoms of a psychiatric illness or medical condition.

### BACKGROUND

Digital mental health has emerged as a promising field that leverages advances in wearable sensor technology and data analytics to remotely monitor and detect various aspects of psychiatric conditions [1]. With a high prevalence of mental health disorders and the increasing global rate of suicide [2], there is a growing need for efficient and unobtrusive methods to identify episodes of illness or medical condition and track their progression over time [3], [4] to enable clinicians to intervene promptly. There have been remote (e-)clinical studies that employ connected devices (e.g., smartphones [5], [6], [7], wearables [8], [9]), as well as customized e-monitoring tools (e.g. mobile apps [10]) to passively collect sensor data and self-rating scale data.

However, one key challenge in the field of digital mental health is the early identification of changes in behavioral and physiological signals that could be predictive of onset of symptoms of a psychiatric illness or medical condition (e.g. a depressive episode in the context of bipolar disorder). Despite the numerous attempts to predict symptom onset accurately, current studies report wide performance heterogeneity across study participants and data quality concerns that introduce spurious artifacts [12]. Moreover, the stochastic nature and nonlinear dynamics of mood regulation [13], [14], make it crucial to provide granular data analysis, rather than relying on summary statistics of windowed and under-sampled data. In fact, the detection and characterization of onset of symptoms is a complex signal processing and pattern recognition task that requires more complex analysis than traditional time-domain methods of anomaly detection and machine learning [17]. While these methods are useful for monitoring and predicting behavioral patterns (e.g. physical activity levels, social interactions) and stress levels using raw data, they often fail to capture the subtle changes associated with the onset of symptoms of mental health conditions, thereby limiting their effectiveness in early detection and intervention.

### SUMMARY

In one aspect, a sensor-based method for individualized prediction of onset of a symptom of a psychiatric illness or medical condition is provided. The method comprises obtaining actimetric patient data from at least one actimetric sensor associated with a patient. The actimetric patient data comprises at least one of time series sleep variable data and time series activity variable data. The method further comprises applying multi-resolution time-frequency analysis to the actimetric patient data to obtain time-frequency spike train data for the patient, predicting onset of the symptom from the time-frequency spike train data, and issuing an alert when the onset of the symptom is predicted from the time-frequency spike train data.

In some embodiments, applying multi-resolution time-frequency analysis to the actimetric patient data to obtain time-frequency spike train data for the patient comprises decomposing the actimetric patient data using empirical mode decomposition to obtain a plurality of temporal modes each representing a respective temporal scale, wherein each temporal mode is represented by an intrinsic mode function. In some particular embodiments, applying multi-resolution time-frequency analysis to the actimetric patient data to obtain time-frequency spike train data for the patient further comprises, after decomposing the actimetric patient data, transforming the intrinsic mode functions to obtain one respective time series of instantaneous frequencies for each one of the intrinsic mode functions. Transforming the intrinsic mode functions to obtain the respective time series of instantaneous frequencies of the intrinsic mode functions may use Hilbert transformation.

In some embodiments, applying multi-resolution time-frequency analysis to the actimetric patient data to obtain time-frequency spike train data for the patient comprises performing spike detection on the instantaneous frequencies. Performing spike detection on the instantaneous frequencies may comprise identifying instantaneous frequencies whose magnitudes exceed respective predetermined limits as spikes representing time-frequency anomalies associated with changes in variability within the actimetric patient data. In some embodiments, predicting onset of the symptom from the time-frequency spike train data comprises computing anomaly scores for the time-frequency spike train data, and may further comprise comparing the anomaly scores to a threshold.

In some embodiments, applying multi-resolution time-frequency analysis to the actimetric patient data to obtain time-frequency spike train data for the patient further comprises, after transforming the intrinsic mode functions to obtain the respective time series of instantaneous frequencies of the intrinsic mode functions, taking a first order derivative of each of the time series of instantaneous frequencies to obtain a spectral derivative time series for each of the intrinsic mode functions.

In some embodiments, applying multi-resolution time-frequency analysis to the actimetric patient data to obtain time-frequency spike train data for the patient further comprises performing spike detection on the spectral derivative time series. Performing spike detection on the spectral derivative time series may comprise identifying spectral derivatives whose magnitudes exceed respective predetermined limits as spikes representing time-frequency anomalies associated with changes in variability within the actimetric patient data. In some embodiments, predicting onset of the symptom from the time-frequency spike train data comprises computing anomaly scores for the time-frequency spike train data, and may further comprise comparing the anomaly scores to a threshold.

In some embodiments, actimetric sensor(s) include at least one body-worn sensor.

Preferably, the actimetric patient data comprises both time series sleep variable data and time series activity variable data.

In some embodiments, the symptom is a depressive episode. In some embodiments where the symptom is a depressive episode, the psychiatric illness or medical condition is one of bipolar disorder or major depressive disorder.

In some embodiments, the symptom is one of a manic episode and a hypomanic episode. In some such embodiments, the psychiatric illness or medical condition is bipolar disorder.

In another aspect, the present disclosure is directed to a computer program product comprising at least one tangible, non-transitory computer-readable medium embodying instructions which, when executed by at least one processor of a data processing system, cause the data processing system to carry out any of the above-described methods.

In yet another aspect, the present disclosure is directed to a system for individualized prediction of onset of a symptom of a psychiatric illness or medical condition, the system comprising at least one actimetric sensor configured to obtain actimetric patient data comprising at least one of time series sleep variable data and time series activity variable data, and at least one data processing system communicatively coupled to the actimetric sensor(s), wherein the at least one data processing system comprises at least one processor and memory coupled to the processor(s), wherein the memory contains instructions which, when implemented by the processor(s), cause the processor(s) to execute any of the above-described methods.

### BRIEF DESCRIPTION OF THE DRAWINGS

These and other features will become more apparent from the following description in which reference is made to the appended drawings wherein:

FIG. 1 is a schematic representation of a first illustrative system for individualized prediction of onset of a symptom of a psychiatric illness or medical condition;

FIG. 1A is a schematic representation of a second illustrative system for individualized prediction of onset of a symptom of a psychiatric illness or medical condition;

FIG. 2 is a flowchart showing an illustrative sensor-based method for individualized prediction of onset of a symptom of a psychiatric illness or medical condition;

FIG. 3A is a flowchart showing sub-steps of a first illustrative method for applying multi-resolution time-frequency analysis to the actimetric patient data;

FIG. 3B is a flowchart showing sub-steps of a second illustrative method for applying multi-resolution time-frequency analysis to the actimetric patient data;

FIG. 4 is a schematic representation of an illustrative sensor-based method for individualized prediction of onset of a symptom of a psychiatric illness or medical condition;

FIG. 5 is a flowchart showing an analytical pipeline for experimental evaluation of a method according to the present disclosure;

FIG. 6 is a graph showing performance metrics for time series activity variable data for an experimental evaluation of a method according to the present disclosure, averaged across study participants;

FIG. 7 is a graph showing performance metrics for time series sleep variable data for an experimental evaluation of a method according to the present disclosure, averaged across study participants;

FIG. 8 is a block diagram of an illustrative computer system in respect of which aspects of the present technology may be implemented; and

FIG. 9 is a block diagram of an illustrative networked mobile wireless telecommunication computing device in the form of a smartphone in respect of which aspects of the present technology may be implemented.

### DETAILED DESCRIPTION

Broadly speaking, the present disclosure describes a sensor-based system and method for individualized prediction of onset of a symptom of a psychiatric illness or medical condition.

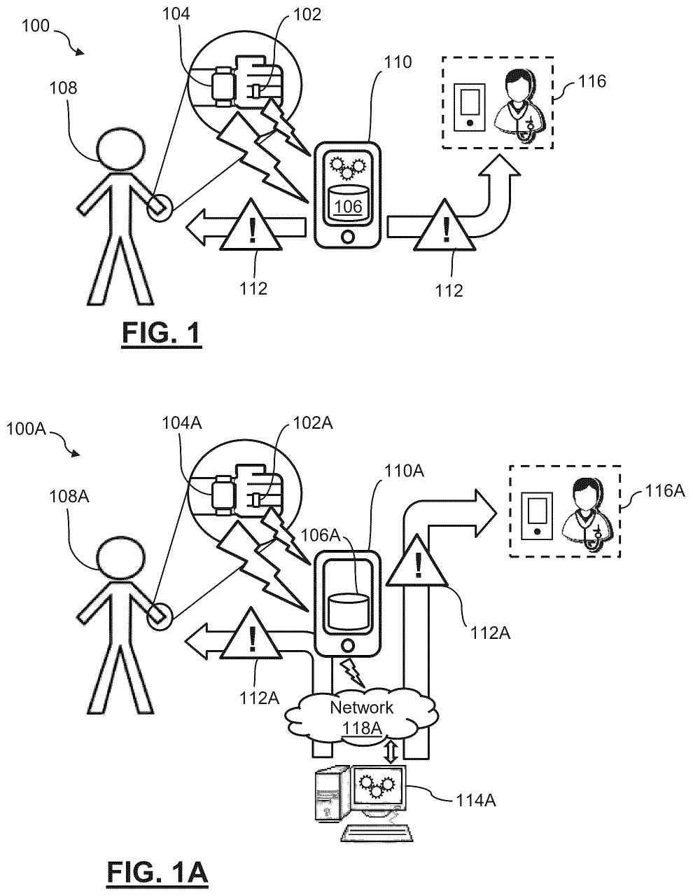

FIG. 1 shows an illustrative, non-limiting embodiment of a system 100 for individualized prediction of onset of a symptom of a psychiatric illness or medical condition. The illustrative system 100 comprises a plurality of actimetric sensors, including an Oura® smart ring 102 and an Apple Watch® smartwatch 104, each of which includes biometric sensor capability. These are merely non-limiting illustrative examples of actimetric sensors; any suitable actimetric sensors may be used. The term “actimetric sensor” thus includes a sensor system comprising a plurality of individual sensor elements. The term “actimetric”, as used herein, refers to the measurement of indicia of the activity of an individual, including exercise, number of steps taken, sleep duration and sleep quality. Although a plurality of actimetric sensors 102, 104 are shown as part of the system of FIG. 1, in other embodiments the system may include only a single actimetric sensor. While the illustrative actimetric sensors are body-worn sensors, other types of actimetric sensors are also contemplated within the scope of the present disclosure.

The actimetric sensors 102, 104 are configured to obtain actimetric patient data 106 from a patient 108. The actimetric patient data comprises at least one of time series sleep variable data and time series activity variable data; preferably the actimetric patient data comprises both time series sleep variable data and time series activity variable data. By way of non-limiting illustrative example, the time series sleep variable data may comprise the number of minutes of deep sleep each day (or within a more granular period, such as each hour), and the time series activity variable data may comprise the number of steps taken each day (or within a more granular period, such as each hour). These are merely examples, and other types of time series actimetric patient data may be used. The time series actimetric patient data is selected based on clinical relevance. Without being limited by theory, it is believed that the number of steps and the number of minutes of deep sleep have high clinical relevance for predicting a depressive episode as a symptom where the psychiatric illness or medical condition is bipolar disorder or major depressive disorder, and for predicting (hypo)manic (i.e. manic or hypomanic) episodes as a symptom where the psychiatric illness or medical condition is bipolar disorder. The foregoing are merely non-limiting examples of symptoms and of psychiatric illness or medical conditiones.

The system 100 further comprises a data processing system communicatively coupled to the actimetric sensors 102, 104. In the illustrated embodiment shown in FIG. 1, the data processing system is a networked mobile wireless telecommunication computing device in the form of a smartphone 110. The smartphone 110 is communicatively coupled to the actimetric sensors 102, 104 wirelessly via Bluetooth; other forms of wireless communicative coupling are also contemplated, and wired communicative coupling is contemplated as well. The smartphone 110 is configured to execute program instructions to apply multi-resolution time-frequency analysis to the actimetric patient data 106 to obtain time-frequency spike train data for the patient 108, and to predict onset of the symptom (e.g. a depressive episode) from the time-frequency spike train data. Illustrative implementations of this process are described further below. The smartphone 110 is further configured to issue an alert 112 when onset of the symptom (e.g. a depressive episode) is predicted from the time-frequency spike train data. The prediction may be based, for example, on an estimated probability of symptom onset exceeding a predetermined threshold. For example, and without limitation, a probability threshold of 0.8 may be used. The alert 112 may be issued directly from the smartphone 110 to alert the patient 108, and/or may be transmitted from the smartphone 110 to the clinical care team 116 for the patient 108, for example by any one or more of e-mail, text message or telephone call. Thus, a member of the clinical care team 116 can initiate appropriate care in advance of onset of the symptom. A bespoke software application may also be used to generate the alert 112. The alert 112 may be, or include, an audible alert as well.

FIG. 1A shows another illustrative, non-limiting embodiment of a system 100A for individualized prediction of onset of a symptom of a psychiatric illness or medical condition, with like reference numerals denoting like features except with the suffix “A”. The system 100A shown in FIG. 1A differs from the system 100 shown in FIG. 1 in that instead of directly performing application of the multi-resolution time-frequency analysis to the actimetric patient data 106A, the smartphone 110A functions as a relay to a second data processing system 114A. In the illustrated embodiment, the second data processing system 114A is a server system 114A, and the smartphone 110A communicates the actimetric patient data 106A to the server system 114A via a network 118A, such as the Internet. For example, the actimetric patient data 106A may be sent from the smartphone 110A to the server system 114A via the network 118A, and/or the smartphone 110A may upload the actimetric patient data 106A into one or more cloud accounts which can then be accessed by the server system 114A, for example using a suitable API. The server system 114A then applies the multi-resolution time-frequency analysis to the actimetric patient data 106A to obtain the time-frequency spike train data for the patient 108A, predicts the onset of the symptom from the time-frequency spike train data and, where such onset is predicted, issues the alert 112A. The alert 112A can be sent by any one or more of e-mail, text message, telephone call or purpose-built application to the clinical care team 116A and optionally to the patient 108A.

In an embodiment such as that shown in FIG. 1A, it is also contemplated that processing steps of application of the multi-resolution time-frequency analysis to the actimetric patient data to obtain the time-frequency spike train data for the patient and/or predicting the onset of the symptom from the time-frequency spike train data may be shared between the smartphone and the server system.

In some embodiments, one or more of the actimetric sensors may be integrated into the data processing system that applies the multi-resolution time-frequency analysis to the actimetric patient data to obtain the time-frequency spike train data for the patient and predicts the onset of the symptom from the time-frequency spike train data, or into the system that relays the actimetric patient data. For example, a smartwatch may include the sensors that obtain the actimetric patient data and may then apply the multi-resolution time-frequency analysis to the actimetric patient data to obtain the time-frequency spike train data for the patient and predict the onset of the symptom from the time-frequency spike train data. A smartwatch may also include cellular capability so as to serve as a relay to communicate the actimetric patient data to a server system via a network such as the Internet, without using an intervening smartphone. In addition, in some embodiments the smartphone may include inbuilt sensors that can be used as actimetric sensors. The smartphone is merely one illustrative embodiment, and in other embodiments the actimetric sensor(s) may be coupled to other types of computer, such as a laptop or desktop computer, for example.

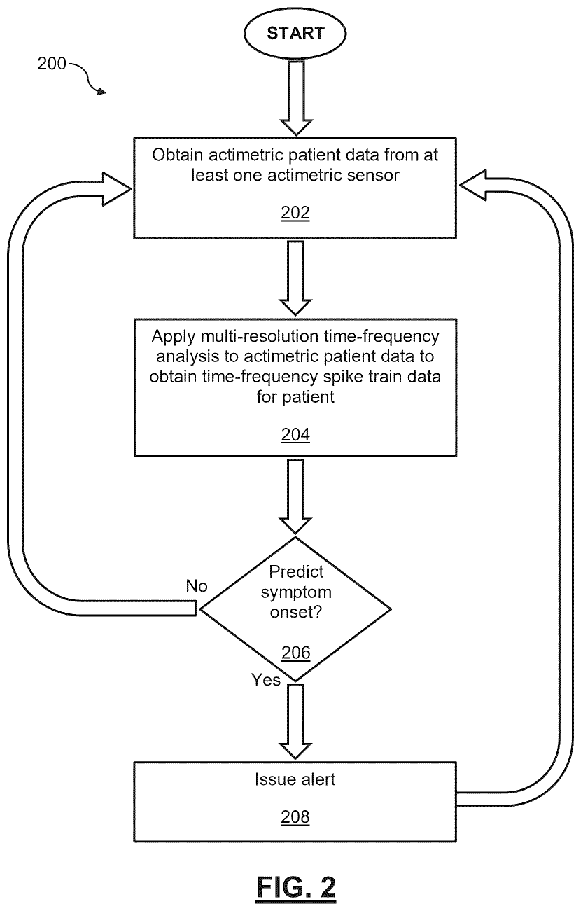

FIG. 2 is a flowchart showing an illustrative sensor-based method 200 for individualized prediction of onset of a symptom (e.g. depressive episode) of a psychiatric illness or medical condition (e.g. bipolar disorder or major depressive disorder). Without being limited by theory, and without limiting their application in the context of other psychiatric illness or medical conditiones, it is believed that the methods described herein have particular application to episodic psychiatric illness or medical conditiones, including mood disorders such as bipolar disorder or major depressive disorder.

At step 202, the method 200 obtains actimetric patient data from at least one actimetric sensor associated with a patient. The actimetric patient data obtained at step 202 comprises at least one of time series sleep variable data and time series activity variable data, and preferably comprises both time series sleep variable data and time series activity variable data. The actimetric patient data obtained at step 202 may, for example, be in the form of a multivariate feature matrix, with each feature being a time series, such as the number of steps taken daily and the number of minutes of deep sleep daily.

At step 204, the method 200 applies multi-resolution time-frequency analysis to the actimetric patient data to obtain time-frequency spike train data for the patient. At step 206, the method 200 predicts the onset of the symptom from the time-frequency spike train data, and at step 208 the method 200 issues an alert when the onset of the symptom is predicted from the time-frequency spike train data (“yes” at step 206). If symptom onset is not predicted (“no” at step 206), or after issuing an alert at step 208, the method 200 returns to step 202 to obtain further actimetric patient data.

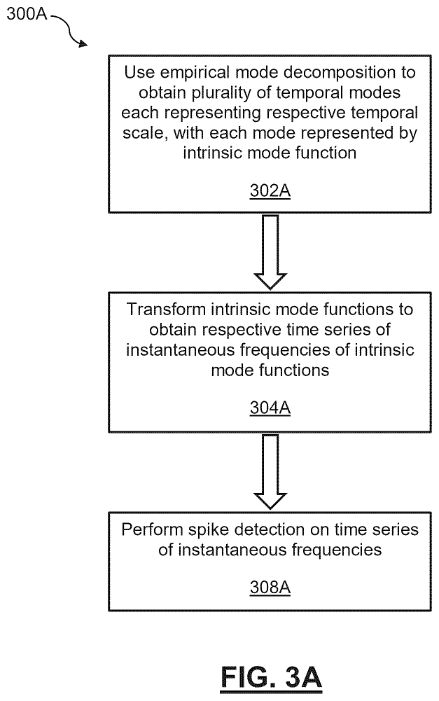

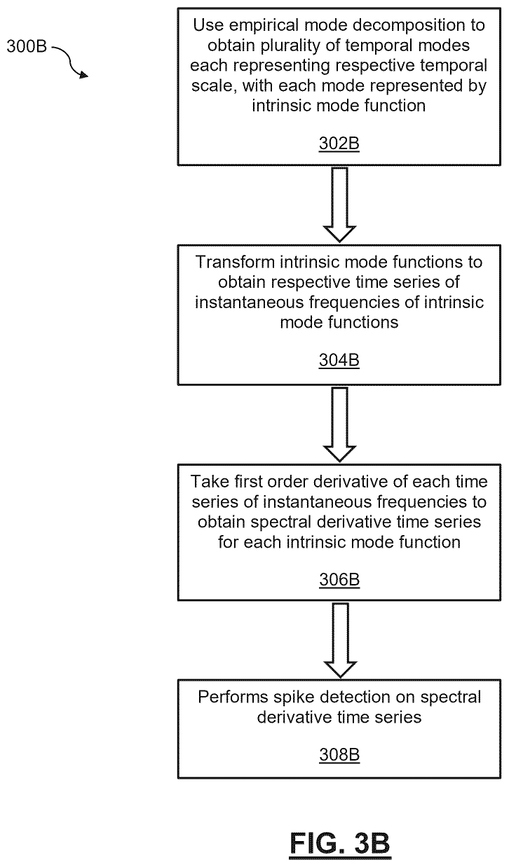

FIG. 3A is a flowchart showing the sub-steps of a first illustrative method 300A for applying the multi-resolution time-frequency analysis to the actimetric patient data at step 204. The method 300A shown in FIG. 3A is used where the time-frequency spike train data used for prediction of the onset of the symptom at step 206 comprises the instantaneous frequencies. At step 302A, empirical mode decomposition is used to obtain a plurality of temporal modes each representing a respective temporal scale. Each temporal mode in turn is represented by an intrinsic mode function. The granularity of the modes is limited by data granularity. For example, if the time series sleep variable data and time series activity variable data is collected every five minutes, the first intrinsic mode function IMF₀ is 5 minutes, the second intrinsic mode function IMF₂ can be hourly, the third intrinsic mode function IMF₃ can be daily, etc. At step 304A, after decomposing the actimetric patient data to obtain the intrinsic mode functions, the intrinsic mode functions are transformed to obtain one respective time series of instantaneous frequencies for each one of the intrinsic mode functions. Step 304A is preferably carried out using Hilbert transformation. At step 308A, the method 300A performs spike detection on the time series of instantaneous frequencies. In a preferred embodiment, performing spike detection on the time series of instantaneous frequencies at step 308A comprises identifying those instantaneous frequencies whose magnitudes exceed respective predetermined limits as spikes representing time-frequency anomalies associated with changes in variability within the actimetric patient data.

FIG. 3B is a flowchart showing the sub-steps of a second illustrative method 300B for applying the multi-resolution time-frequency analysis to the actimetric patient data at step 204. The method 300B shown in FIG. 3B is used where the time-frequency spike train data used for prediction of the onset of the symptom at step 206 comprises spectral derivatives. Steps 302B and 304B of the method 300B shown in FIG. 3B are identical to steps 302A and 304A of the method 300A shown in FIG. 3A. However, in the method 300B shown in FIG. 3B, an additional step 306A is carried out before spike detection. After transforming the intrinsic mode functions to obtain the respective time series of instantaneous frequencies of the intrinsic mode functions at step 304A, at step 306B the method 300B takes a first order derivative of each of the time series of instantaneous frequencies to obtain a respective spectral derivative time series for each of the intrinsic mode functions. The term “spectral”, as used herein, refers to the time-varying nature of the instantaneous frequencies, which exist along a range analogously to time-varying wavelengths of light (color) in a flame. Thus, a spectral derivative captures the rate of variation of the instantaneous frequencies, analogously to how spectral analysis of a flame could capture the rate of variation in its color (variation in light wavelength on the electromagnetic spectrum). Thus, the term “spectral” describes the context in which the derivative is taken, and one could equivalently omit the word “spectral” and refer to taking a first order derivative of each of the time series of instantaneous frequencies to obtain a respective first order derivative time series for each of the intrinsic mode functions. Then, at step 308B the method 300B performs spike detection on the spectral derivative time series. Preferably, spike detection on the spectral derivative time series comprises identifying those spectral derivatives whose magnitudes exceed respective predetermined limits as spikes representing time-frequency anomalies associated with changes in variability within the actimetric patient data.

Returning to FIG. 2, predicting the onset of the symptom from the time-frequency spike train data at step 206 comprises identifying any spikes within a predetermined period (either instantaneous frequency spikes as per the method 300A in FIG. 3A or spectral derivative spikes as per the method 300B in FIG. 3B), relative to a spike baseline. Preferably, identification of spikes within the predetermined period is done individually for each intrinsic mode function, and then a probability of symptom onset can be calculated using a composite weighted prediction approach, an illustrative embodiment of which is described further below.

Thus, the present disclosure describes methods that apply time-frequency analysis, preferably augmented with spectral derivative techniques, to enable detection and localization of clinically relevant patterns in activity and sleep data collected from individuals. By leveraging the multi-resolution nature of time-frequency analysis, methods according to the present disclosure enable the identification of both sudden deceleration and sudden acceleration of passively collected activity and sleep data, providing a comprehensive view of the behavioral changes associated with onset of symptoms of a psychiatric illness or medical condition.

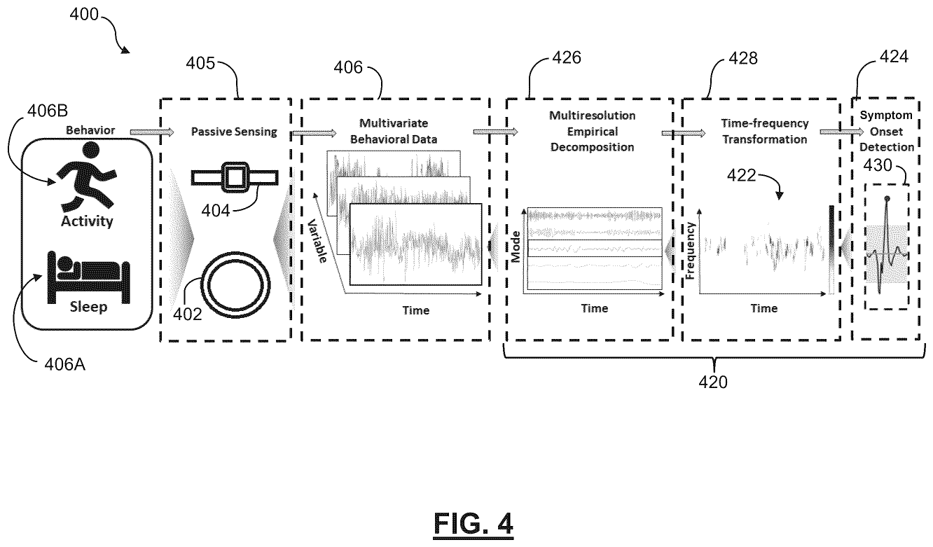

Reference is now made to FIG. 4, which is a schematic representation of an illustrative sensor-based method 400 for individualized prediction of onset of a symptom (e.g. depressive episode) of a psychiatric illness or medical condition (e.g. bipolar disorder or major depressive disorder). FIG. 4 provides context for a more detailed mathematical explanation of illustrative implementations of the method 400.

The method 400 obtains actimetric patient data 406 from at least one actimetric sensor 402, 404 associated with a patient, preferably by way of passive sensing 405. As noted above, the actimetric patient data 406 comprises at least one of time series sleep variable data 406A and time series activity variable data 406B, and preferably both time series sleep variable data 404A and time series activity variable data 404B.

The method 400 applies multi-resolution time-frequency analysis 420 to the actimetric patient data 406 to obtain time-frequency spike train data 422 for the patient, and then predicts 424 onset of the symptom from the time-frequency spike train data 422.

Application of the multi-resolution time-frequency analysis 420 to the actimetric patient data 406 to obtain the time-frequency spike train data comprises decomposing the actimetric patient data using empirical mode decomposition 426 (abbreviated “EMD”) to obtain a plurality of temporal modes each representing a respective temporal scale. The temporal modes may be, for example, monthly variability, weekly variability, daily variability and hourly variability, depending on the granularity of the data. Each temporal mode is represented by an intrinsic mode function.

In an illustrative embodiment, multivariate oscillatory mode decomposition is performed by sifting, defined as the process of cubic spline interpolation of the local extrema of each input variable. The Fourier and Wavelet transforms are inherently limited in terms of time-frequency resolution by the a priori choice of the basis functions (e.g., cosine, Morlet mother wavelet). Instead, a preferred embodiment of empirical mode decomposition 426 uses empirical decomposition of the intrinsic modes of the input data with no a priori stationarity or pattern templates and is data-driven [21], without assuming input time series stationarity. Although the original empirical mode decomposition algorithm may be used, it suffers from the mode-mixing problem caused by intermittency, manifested in a single intrinsic mode function carrying data from different scales, or a single mode pattern distributed across different intrinsic mode functions. Therefore, a preferred embodiment uses the Complete Ensemble EMD with Additive Noise (CEEMDAN) algorithm for empirical mode decomposition. Other empirical mode decomposition methods may be used, but are less preferred, relative to CEEMDAN. CEEMDAN is based on repetitively adding variable and finite amplitudes of Gaussian white noise to the input signal, decomposing the resulting signal into intrinsic mode functions, and computing the ensemble average of the realizations for each intrinsic mode function. The ensemble averaged intrinsic mode functions maintain their natural properties while minimizing the probability of mode mixing. The fully optimized CEEMDAN algorithm is formulated in equations (1) to (5):

$${IMF}_{1} = 1 / I {\sum }_{i = 1}^{I} {E}_{1} [ x + ε {w}_{i} ] \qquad ( 1 )$$

where IMF₁ is the first empirical mode decomposition component of the input signal x, I is the number of realizations, wᵢ is the added zero-mean unit-variance Gaussian white noise, ε is a constant coefficient determining the noise level relative to the input signal's standard deviation, and Eᵢ is the function that generates the intrinsic mode function.

$${r}_{1} = x - {IMF}_{1} \qquad ( 2 )$$

$${IMF}_{( k + 1 )} = 1 / I {\sum }_{i = 1}^{1} {E}_{1} [ {r}_{k} + ε {E}_{k} [ {w}_{i} ] ] \qquad ( 3 )$$

$$R = x - {\sum }_{i = 1}^{I} {IMF}_{k} \qquad ( 4 )$$

$$x = {\sum }_{i = 1}^{I} {IMF}_{k} + R \qquad ( 5 )$$

where R is the final residual, and K is the number of decomposed intrinsic mode functions, such that any input signal x can be completely reconstructed with minimal mode mixing.

After decomposing the actimetric patient data, the intrinsic mode functions are transformed 428 to obtain one respective time series of instantaneous frequencies for each one of the intrinsic mode functions. Preferably, the Hilbert transformation is used. Each intrinsic mode function has its own time series of instantaneous frequencies.

The instantaneous frequency and amplitude of each intrinsic mode function is given by equations (6) and (7):

$$A [ n ] = \sqrt{{x}^{2} [ n ] + {y}^{2} [ n ]} \qquad ( 6 )$$

$$IF [ n ] = ( 1 / 2 π ) \frac{x [ n ] {y}^{′} [ n ] - {x}^{′} [ n ] y [ n ]}{{x [ n ]}^{2} + {y [ n ]}^{2}} \qquad ( 7 )$$

where IF[n] is the instantaneous frequency, x[n] is the separated intrinsic mode function, x′[n] is the derivative of x[n] with respect to n, A[n] is the instantaneous amplitude of the separated intrinsic mode function, y[n] is the Hilbert transform of x[n], y′[n] is the derivative of y[n] with respect to n, and tₛ is the sampling interval.

As noted above in the context of FIGS. 2 and 3A, in one embodiment the time-frequency spike train data 422 used for prediction of the onset of the symptom comprises the instantaneous frequencies. In such embodiments, application of the multi-resolution time-frequency analysis 420 to the actimetric patient data 406 to obtain the time-frequency spike train data comprises performing spike detection 430 on the instantaneous frequencies. In one preferred embodiment, spike detection 430 may be performed on the instantaneous frequencies by identifying those instantaneous frequencies whose magnitudes exceed respective predetermined limits as spikes representing time-frequency anomalies (e.g. increases or decreases) associated with changes in variability within the actimetric patient data. Thus, a spike would be identified where equation (8) is true:

$$( IF [ n ] < LB ) or ( IF [ n ] > UB ) \qquad ( 8 )$$

where LB is a lower bound and UB is an upper bound.

The upper and lower bounds are statistical thresholds to assess the instantaneous frequency values. By mathematically comparing the instantaneous frequency, denoted as IF[n], with the confidence interval, delimited by the lower bound and the upper bound, instances where the instantaneous frequency lies outside the confidence interval can be identified as spikes. In one preferred embodiment, the confidence interval is 90% but this is merely illustrative and not limiting. The upper and lower bounds may be different, and may be determined empirically. For example, the upper and lower bounds may be based on a number of standard deviations, or a confidence interval. Equivalently, the absolute value of the instantaneous frequency may be compared to a single limit value.

While the instantaneous frequencies may be used to predict the onset of the symptom, in a preferred embodiment a spectral derivative is used, as described above in the context of FIGS. 2 and 3B. In this preferred embodiment, instead of performing spike detection using equation (8), after transforming the intrinsic mode functions to obtain the respective time series of instantaneous frequencies of the intrinsic mode functions, equation (9) is used to take a first order derivative of each of the time series of instantaneous frequencies to obtain a respective spectral derivative time series for each of the intrinsic mode functions. Each time series of instantaneous frequencies has its own spectral derivative, so each intrinsic mode function has its own corresponding time-varying spectral derivative. Equation (9) is given below.

$$SD [ n ] = \frac{IF [ n ] - IF [ n - 1 ]}{{t}_{s}} \qquad ( 9 )$$

where SD[n] denotes the spectral derivative of the instantaneous frequency IF[n].

Thus, in this embodiment, the time-frequency spike train data 422 used for prediction of the onset of the symptom comprises the spectral derivative time series, and spike detection 430 can then be performed on the spectral derivative time series. In a preferred embodiment, spike detection 430 is performed on the spectral derivative time series by identifying spectral derivatives whose magnitudes exceed respective predetermined limits as spikes representing time-frequency anomalies associated with changes (e.g. increases or decreases) in variability within the actimetric patient data. Thus, a spike would be identified where equation (10) is true:

$$( SD [ n ] < LB ) or ( SD [ n ] > UB ) \qquad ( 10 )$$

where LB is a lower bound and UB is an upper bound.

The upper and lower bounds are statistical thresholds to assess the spectral derivative values. By mathematically comparing the spectral derivative, denoted as SD[n], with the confidence interval as delimited by the upper and lower bounds, instances where the spectral derivative lies outside the confidence interval can be identified as spikes. In one preferred embodiment, the confidence interval is 90% but this is merely illustrative and not limiting. The upper and lower bounds may be different, and may be determined empirically. For example, the upper and lower bounds may be based on a number of standard deviations, or a confidence interval. Equivalently, the absolute value of the spectral derivative may be compared to a single limit value.

Spike detection 430 can be performed by comparing either the instantaneous frequencies or the spectral derivatives to a spike baseline. Preferably, this is done individually for each intrinsic mode function. The spike baseline can be established by analyzing an initial set of actimetric patient data to obtain the instantaneous frequencies or the spectral derivatives using the techniques described above, and then comparing this initial dataset to the patient's self-reporting of symptoms. Self-reporting may be obtained using suitable questionnaires, such as (for example) the Patient Health Questionnaire (PHQ-9) [18] for depressive symptoms and the Altman Self Rating Mania Scale (ASRS) [23] for hypomanic or manic symptoms. These are merely non-limiting, illustrative examples of suitable questionnaires. Suitable statistical analysis can then be performed, including measurement of false positives, true positives, false negatives and true negatives, to establish the baseline for that particular patient. The spike baseline may be a range having upper and lower limits. In such an embodiment, if, within a predetermined period, any of the instantaneous frequencies or the spectral derivatives exceeds the upper limit, or falls below the lower limit, onset of the symptom would be predicted. If the instantaneous frequencies or the spectral derivatives are all between the upper and lower limits, this would be considered to be within a range of normal variability.

One illustrative method for determining a baseline for performing spike detection using instantaneous frequencies will now be described.

The actimetric patient data 406 comprises time series activity variable data 406B, represented as X(t), and time series sleep variable data 406A, represented as Y(t). The actimetric patient data 406 is segmented into N time windows, each of length T. Then, Xᵢt) and Yᵢ(t) are the time series data for the i^{th} time window, and

$${IMX}_{i}^{k} ( t ) and {IMY}_{i}^{k} ( t )$$

are the k^{th} intrinsic mode functions (IMFs) obtained from empirical mode decomposition 426, using the CEEMDAN algorithm applied to Xᵢ(t) and Yᵢ(t), respectively.

Separate outlier detection models are trained for the instantaneous frequencies of selected intrinsic mode functions from both time series activity variable data 406B, represented as X(t), and time series sleep variable data 406A, represented as Y(t). These models assign anomaly scores to each time window, reflecting deviations from learned normal patterns. Higher anomaly scores indicate a greater likelihood of the data point being an outlier, i.e., predictive of symptom onset.

$${A}_{{IMX}_{i}^{k}} and {A}_{{IMY}_{i}^{k}}$$

represent the anomaly scores for the k^{th} intrinsic mode function from activity and sleep data in the i^{th} time window and

$${ω}_{{IMX}_{i}^{k}} ( t ) and {ω}_{{IMY}_{i}^{k}} ( t )$$

represent the instantaneous frequencies of the k^{th} intrinsic mode function obtained using the Hilbert transform. The anomaly scores

$${A}_{{IMX}_{i}^{k}} and {A}_{{IMY}_{i}^{k}}$$

are calculated by comparing the instantaneous frequencies

$${ω}_{{IMX}_{i}^{k}} ( t ) and {ω}_{{IMY}_{i}^{k}} ( t )$$

against the learned distribution of normal patterns, as follows.

First, the mean and standard deviation of normal patterns (i.e., during periods where there are no symptoms, or to subsyndromal symptoms) is determined.

For each intrinsic mode function for activity, the mean μ_{IMX}_{ₖ} and standard deviation σ_{IMX}_{ₖ} of the instantaneous frequencies ω_{IMX}_{ₖ}(t) are calculated from a training set of normal (non-anomalous) activity data:

$${μ}_{{IMX}_{k}} = \frac{1}{N} {\sum }_{i = 1}^{N} {ω}_{{IMX}_{k}} ( t ) \qquad ( 11 )$$

$${σ}_{{IMX}_{k}} = \sqrt{\frac{1}{N} {\sum }_{i = 1}^{N} {( {ω}_{{IMX}_{i}^{k}} ( t ) - {ω}_{{IMX}_{k}} )}^{2}} \qquad ( 12 )$$

Similarly, for each intrinsic mode function for sleep, the mean μ_{IMX}_{ₖ} and standard deviation σ_{IMX}_{ₖ} of the instantaneous frequencies ω_{IMX}_{ₖ}(t) are calculated from the training set of normal (non-anomalous) sleep data:

$${μ}_{{IMY}_{k}} = \frac{1}{N} {\sum }_{i = 1}^{N} {ω}_{{IMY}_{k}} ( t ) \qquad ( 13 )$$

$${σ}_{{IMY}_{k}} = \sqrt{\frac{1}{N} {\sum }_{i = 1}^{N} {( {ω}_{{IMY}_{i}^{k}} ( t ) - {ω}_{{IMY}_{k}} )}^{2}} \qquad ( 14 )$$

The same time window is used for both the activity data and the sleep data.

The anomaly scores for the k^{th} intrinsic mode function in the i^{th} time window are then calculated as the z-scores of the instantaneous frequencies

$${ω}_{{IMX}_{i}^{k}} ( t ) and {ω}_{{IMY}_{i}^{k}} ( t ) :$$

$${A}_{{IMX}_{i}^{k}} = \frac{{ω}_{{IMX}_{i}^{k}} ( t ) - {μ}_{{IMX}_{k}}}{{σ}_{{IMX}_{k}}} \qquad ( 15 )$$

$${A}_{{IMY}_{i}^{k}} = \frac{{ω}_{{IMY}_{i}^{k}} ( t ) - {μ}_{{IMY}_{k}}}{{σ}_{{IMY}_{k}}} \qquad ( 16 )$$

These z-scores

$${A}_{{IMX}_{i}^{k}} and {A}_{{IMY}_{i}^{k}}$$

represent the distance, measured in standard deviations, of a data point from the mean of the normal distribution. Higher absolute values of the z-scores indicate a higher likelihood of the data point being an anomaly.

Once the anomaly scores for each intrinsic mode function are computed, they can be combined to form a composite anomaly score. Weights

$${α}_{IMX}^{k} and {β}_{IMY}^{k}$$

are assigned to the k^{th} intrinsic mode function for activity and sleep data, respectively. These weights vary across fields and applications based on clinical relevance. Suitable weighting is within the capability of one of ordinary skill in the art, now informed by the present disclosure. The composite anomaly score Cᵢ b for the i^{th} time window is calculated as:

$${C}_{i} = {\sum }_{k = 1}^{K} {α}_{IMX}^{k} \cdot {A}_{{IMX}_{i}^{k}} + {\sum }_{k = 1}^{K} {β}_{IMY}^{k} \cdot {A}_{{IMY}_{i}^{k}} \qquad ( 17 )$$

Statistical analysis of the composite anomaly scores begins with separating the scores by label. Let Cₚₒₛ be the set of composite anomaly scores for time windows with symptom onset, defined by two or more consecutive weeks of self-reported PHQ-9 scores greater than or equal to 10. Then, for time windows with symptom onset, the mean and standard deviation of the composite anomaly scores are calculated. Using the sliding window approach, there may be some time windows that have different labels associated with different weeks. For example, a one-week window that “slides” daily may overlap one week that has a “1” label and another week that has a “0” label. To address this scenario, the mode (the most frequent label for that time window) may be used. Thus, if a one-week window has four days with a “1” label and three days with a “0” label, that one-week window will be assigned a label of “1”.

The mean composite anomaly score is given by:

$${μ}_{pos} = \frac{1}{{N}_{pos}} {\sum }_{i = 1}^{{N}_{pos}} {C}_{pos , i} \qquad ( 18 )$$

where Nₚₒₛ is the number of time windows with mood episodes, and C_{pos,i} is the composite anomaly score for the i^{th} time window with a mood episode.

The standard deviation of composite anomaly scores is given by:

$${σ}_{pos} = \sqrt{\frac{1}{{N}_{pos}} {\sum }_{i = 1}^{{N}_{pos}} {( {C}_{pos , i} - {μ}_{pos} )}^{2}} \qquad ( 19 )$$

Then, the threshold t is based on the distribution of Cₚₒₛ and a statistical thresholding approach is employed, as follows:

$$τ = {μ}_{pos} + k \cdot {σ}_{pos} \qquad ( 20 )$$

where k is a constant reflecting the desired confidence level (e.g., 1.96 for a 95% confidence interval). Selection of a suitable confidence interval is within the capability of one skilled in the art.

Once the thresholds are set based on historical data analysis, they can function as a spike baseline and the system can be used for real-time prediction of symptom onset.

The actimetric patient data 406, comprising time series activity variable data 406B and time series sleep variable data 406A, is collected continuously in real time. Then, sliding-window real-time instantaneous frequencies are computed. For each new time window, for example a one-week time window where the inter-spike interval is two weeks, the instantaneous frequencies

$${ω}_{IMX}^{k} ( t ) and {ω}_{IMY}^{k} ( t )$$

for the k^{th} intrinsic mode function are computed; these frequencies will be used to calculate sliding-window real-time anomaly scores.

For each new time window, the z-scores (anomaly scores) are computed based on the baseline mean and standard deviation to enable deviations from normal behavior to be quantified in real-time:

$${A}_{{IMX}_{i}^{k}} = \frac{{ω}_{{IMX}_{i}^{k}} ( t ) - {μ}_{{IMX}_{k}}}{{σ}_{{IMX}_{k}}} \qquad ( 21 )$$

$${A}_{{IMY}_{i}^{k}} = \frac{{ω}_{{IMY}_{i}^{k}} ( t ) - {μ}_{{IMY}_{k}}}{{σ}_{{IMY}_{k}}} \qquad ( 22 )$$

Next, sliding-window real-time composite anomaly scores are computed. Combine the weighted anomaly scores from all intrinsic mode functions to compute the composite anomaly score Cᵢ for the current time window:

$${C}_{i} = {\sum }_{k = 1}^{K} {α}_{IMX}^{k} \cdot {A}_{{IMX}_{i}^{k}} + {\sum }_{k = 1}^{K} {β}_{IMY}^{k} \cdot {A}_{{IMY}_{i}^{k}} \qquad ( 23 )$$

This integrates multiple aspects of the data to get a comprehensive measure of anomaly likelihood.

Real-time thresholding can then be carried out by comparing the real-time composite anomaly score Cᵢ with the pre-determined threshold τ. If Cᵢ>τ, the time window is flagged as a potential symptom onset. If Cᵢ<τ, the time window is considered normal.

The above technique may be adapted for spectral derivatives rather than instantaneous frequencies. The techniques and equations used for spectral derivatives rather than instantaneous frequencies are similar, and thus in the discussion and equations that follow, a “prime” indicator used to distinguish between the equations and expressions used for spectral derivatives and those used for instantaneous frequencies. For example,

$${A}_{{IMX}_{i}^{k}} and {A}_{{IMY}_{i}^{k}}$$

(no prime) represent the anomaly scores for the k^{th} intrinsic mode function from activity and sleep data in the i^{th} time window for instantaneous frequencies, and

$${A}_{{IMX}_{i}^{k}}^{′} and {A}_{{IMY}_{i}^{k}}^{′}$$

(with prime) represent the anomaly scores for the k^{th} intrinsic mode function from activity and sleep data in the i^{th} time window for spectral derivatives. For the avoidance of doubt, unlike in equation (7) where a “prime” was used to indicate a derivative, in the discussion and equations that follow, the “prime” indicator for the equations and expressions used for spectral derivatives does not indicate a derivative of the corresponding equations and expressions used for instantaneous frequencies but merely distinguishes between them.

As in the embodiment for instantaneous frequencies, in the embodiment for spectral derivatives the actimetric patient data 406 comprises time series activity variable data 406B, represented as X(t), and time series sleep variable data 406A, represented as Y(t), segmented into N time windows, each of length T. Then, Xᵢ(t) and Yᵢ(t) are the time series data for the i^{th} time window, and

$${IMX}_{i}^{k} ( t ) and {IMY}_{i}^{k} ( t )$$

are the k^{th} intrinsic mode functions (IMFs) obtained from empirical mode decomposition 426, using the CEEMDAN algorithm applied to Xᵢ(t) and Yᵢ(t), respectively.

Separate outlier detection models are trained for the spectral derivatives of selected intrinsic mode functions from both time series activity variable data 406B, represented as X(t), and time series sleep variable data 406A, represented as Y(t). These models assign anomaly scores to each time window to reflect deviations from learned normal patterns, with higher anomaly scores indicating a greater likelihood that a data point is an outlier and therefore predictive of symptom onset.

$${A}_{{IMX}_{i}^{k}}^{′} and {A}_{{IMY}_{i}^{k}}^{′}$$

represent the anomaly scores for the k^{th} intrinsic mode function from activity and sleep data in the i^{th} time window and

$${ω}_{{IMX}_{i}^{k}}^{′} ( t ) and {ω}_{{IMY}_{i}^{k}}^{′} ( t )$$

represent the spectral derivatives (i.e., first-order derivatives of the instantaneous frequencies

$${ω}_{{IMX}_{i}^{k}}^{′} ( t ) and {ω}_{{IMY}_{i}^{k}}^{′} ( t )$$

for the k^{th} intrinsic mode function obtained using the Hilbert transform. The anomaly scores

$${A}_{{IMX}_{i}^{k}}^{′} and {A}_{{IMY}_{i}^{k}}^{′}$$

are calculated by comparing the spectral derivatives

$${ω}_{{IMX}_{i}^{k}}^{′} ( t ) and {ω}_{{IMY}_{i}^{k}}^{′} ( t )$$

(rather than the instantaneous frequencies

$${ω}_{{IMX}_{i}^{k}} ( t ) and {ω}_{{IMY}_{i}^{k}} ( i )$$

themselves, against the learned distribution of normal patterns, as follows.

First, the mean and standard deviation of normal patterns of the spectral derivatives (i.e., during periods where there are no symptoms, or to subsyndromal symptoms) are calculated.

For each intrinsic mode function for activity, the mean μ′_{IMX}_{ₖ}, and standard deviation σ′_{IMX}_{ₖ} of the spectral derivatives ω′_{IMX}_{ₖ}(t) are calculated from a training set of normal (non-anomalous) activity data:

$${μ}_{{IMX}_{k}}^{′} = \frac{1}{N} {\sum }_{i = 1}^{N} {ω}_{{IMX}_{k}}^{′} ( t ) \qquad ( 24 )$$

$${σ}_{{IMX}_{k}}^{′} = \sqrt{\frac{1}{N} {\sum }_{i = 1}^{N} {( {ω}_{{IMX}_{i}^{k}}^{′} ( t ) - {ω}_{{IMX}_{k}}^{′} )}^{2}} \qquad ( 25 )$$

Similarly, for each intrinsic mode function for sleep, the mean μ′_{IMX}_{ₖ} and standard deviation σ′_{IMX}_{ₖ} of the spectral derivatives ω′_{IMX}_{ₖ}(t) are calculated from the training set of normal (non-anomalous) activity data:

$${μ}_{{IMY}_{k}}^{′} = \frac{1}{N} {\sum }_{i = 1}^{N} {ω}_{{IMY}_{k}}^{′} ( t ) \qquad ( 26 )$$

$${σ}_{{IMY}_{k}}^{′} = \sqrt{\frac{1}{N} {\sum }_{i = 1}^{N} {( {ω}_{{IMY}_{i}^{k}}^{′} ( t ) - {ω}_{{IMY}_{k}}^{′} )}^{2}} \qquad ( 27 )$$

As before, the same time window is used for both the activity data and the sleep data.

The anomaly scores for the k^{th} intrinsic mode function in the i^{th} time window are then calculated as the z-scores of the spectral derivatives

$${ω}_{{IMX}_{i}^{k}}^{′} ( t ) and {ω}_{{IMY}_{i}^{k}}^{′} ( t ) :$$

$${A}_{{IMX}_{i}^{k}}^{′} = \frac{{ω}_{{IMX}_{i}^{k}}^{′} ( t ) - {μ}_{{IMX}_{k}}^{′}}{{σ}_{{IMX}_{k}}^{′}} \qquad ( 28 )$$

$${A}_{{IMY}_{i}^{k}}^{′} = \frac{{ω}_{{IMY}_{i}^{k}}^{′} ( t ) - {μ}_{{IMY}_{k}}^{′}}{{σ}_{{IMY}_{k}}^{′}} \qquad ( 29 )$$

These z-scores

$${A}_{{IMX}_{i}^{k}}^{′} and {A}_{{IMY}_{i}^{k}}^{′}$$

represent the distance of a data point from the mean of the normal distribution, measured in standard deviations. Higher absolute values of the z-scores indicate a higher likelihood of the data point being an anomaly.

After the anomaly scores for each intrinsic mode function have been computed, they can be combined to form a composite anomaly score. Weights

$${α}_{IMX}^{′ k} and {β}_{IMY}^{′ k}$$

are assigned to the k^{th} intrinsic mode function for activity and sleep data, respectively. These weights vary across fields and applications based on clinical relevance, with suitable weighting being within the capability of one of ordinary skill in the art, now informed by the present disclosure. The composite anomaly score C′ᵢ b for the i^{th} time window is calculated as:

$${C}_{i}^{′} = {\sum }_{k = 1}^{K} {α}_{IMX}^{′ k} \cdot {A}_{{IMX}_{i}^{k}}^{′} + {\sum }_{k = 1}^{K} {β}_{IMY}^{′ k} \cdot {A}_{{IMY}_{i}^{k}}^{′} \qquad ( 30 )$$

The composite anomaly scores are then separating the scores by label. C′ₚₒₛ is the set of composite anomaly scores for time windows with symptom onset (as noted above, in the illustrated embodiment this is defined by two or more consecutive weeks of self-reported PHQ-9 scores greater than or equal to 10). Then, for time windows with symptom onset, the mean and standard deviation of the composite anomaly scores are calculated (as for the instantaneous frequencies, where there are time windows that have different labels associated with different weeks, the mode (the most frequent label for that time window) may be used.

The mean composite anomaly score is given by:

$${μ}_{pos}^{′} = \frac{1}{{N}_{pos}^{′}} {\sum }_{i = 1}^{{N}_{pos}^{′}} {C}_{pos , i}^{′} \qquad ( 31 )$$

where N′ₚₒₛ is the number of time windows with mood episodes, and C′_{pos,i} is the composite anomaly score for the i^{th} time window with a mood episode.

The standard deviation of composite anomaly scores is given by:

$${σ}_{pos}^{′} = \sqrt{\frac{1}{{N}_{pos}^{′}} {\sum }_{i = 1}^{{N}_{pos}^{′}} {( {C}_{pos , i}^{′} - {μ}_{pos}^{′} )}^{2}} \qquad ( 32 )$$

Then, the threshold τ′ is based on the distribution of C′ₚₒₛ and a statistical thresholding approach is employed, as follows:

$${τ}^{′} = {μ}_{pos}^{′} + {k}^{′} \cdot {σ}_{pos}^{′} \qquad ( 33 )$$

where k′ is a constant reflecting the desired confidence level. Selection of a suitable confidence interval is within the capability of one skilled in the art.

Once set based on historical data analysis, the thresholds can function as a spike baseline and the system can be used for real-time prediction of symptom onset.

The actimetric patient data 406, comprising time series activity variable data 406B and time series sleep variable data 406A, is collected continuously in real time. Then, sliding-window real-time spectral derivatives are computed. For each new time window, the spectral derivatives

$${ω}_{IMX}^{′ k} ( t ) and {ω}_{IMY}^{′ k} ( t )$$

for the k^{th} intrinsic mode function are computed and used to calculate sliding-window real-time anomaly scores.

For each new time window, the z-scores (anomaly scores) are computed based on the baseline mean and standard deviation so that deviations from normal behavior can be quantified in real-time:

$${A}_{{IMX}_{i}^{k}}^{′} = \frac{{ω}_{{IMX}_{i}^{k}}^{′} ( t ) - {μ}_{{IMX}_{k}}^{′}}{{σ}_{{IMX}_{k}}^{′}} \qquad ( 34 )$$

$${A}_{{IMY}_{i}^{k}}^{′} = \frac{{ω}_{{IMY}_{i}^{k}}^{′} ( t ) - {μ}_{{IMY}_{k}}^{′}}{{σ}_{{IMY}_{k}}^{′}} \qquad ( 35 )$$

Sliding-window real-time composite anomaly scores are then computed, and the weighted anomaly scores from all intrinsic mode functions are combined to compute the composite anomaly score Cᵢ for the current time window:

$${C}_{i}^{′} = {\sum }_{k = 1}^{K} {α}_{IMX}^{′ k} \cdot {A}_{{IMX}_{i}^{k}}^{′} + {\sum }_{k = 1}^{K} {β}_{IMY}^{′ k} \cdot {A}_{{IMY}_{i}^{k}}^{′} \qquad ( 36 )$$

The composite anomaly score Cᵢ integrates multiple aspects of the data to get a comprehensive measure of anomaly likelihood.

Real-time thresholding can then be carried out by comparing the real-time composite anomaly score C′ᵢ with the pre-determined threshold τ′. If C′ᵢ>τ′, the time window is flagged as a potential symptom onset. If C′ᵢ<τ′, the time window is considered normal.

Thus, positive and negative peaks in the instantaneous frequencies or spectral derivatives, occurring outside the confidence interval, signify probable symptom onset. When these peaks are identified, a flag can be set to indicate the onset of a symptom (e.g. depressive episode), facilitating prompt clinical intervention. In a preferred embodiment, any spike (anomaly) detected within a predetermined period (relative to a spike baseline) is considered to be a predicted symptom onset.

### Experimental Results

### Dataset

Data from 145 participants, of whom 92 (63.4%) were diagnosed with bipolar disorder type I and 53 (36.6%) were diagnosed with bipolar disorder type II, was analyzed. The participants were enrolled in the study for a duration of 449±224 days until Jun. 23, 2023. Participants were provided with an Ōura® smart ring, Generation 2, a wearable ring that continuously measures activity (e.g., number of steps), sleep (e.g., total sleep duration), and cardiorespiratory variables (e.g., heart rate). The participants were mailed a sizing kit for individualized sensor size selection to ensure optimal skin-sensor contact and data quality. Additionally, participants received a secure e-mailed link asking them to complete a weekly Patient Health Questionnaire (PHQ-9) and Altman Self-Rating Mania Scale (ASRS) through a secure email link. Participants were required to complete all items on both self-rating scales to submit their ratings. The number of daily steps was selected as a representative variable for the time series activity variable data. The number of minutes of total sleep per night was selected as a representative variable for the time series sleep variable data. This data was collected at an average of 33.8±47.2 days of non-wear per participant.

### Data Quality Assessment and Control

To accommodate variations in activity and sleep among participants and the fact that the sleep variables and the activity variables each use different metrics, the time series activity variable data and the time series sleep variable data were normalized within a range of 0 to 1. Once normalized, each participant's data were categorized into two binary labels. Euthymic (i.e., stable mood) data were labeled as ‘0’, while data indicative of a depressive episode were labeled as ‘1’. The labels were applied to the time series activity variable data and the time series sleep variable data based on whether the data occurred during a euthymic period or a depressive episode, as indicated by the weekly reported total PHQ-9 score, calculated as the sum of the nine (9) individual item scores. A depressive episode was clinically defined as a period lasting at least two consecutive weeks with a total PHQ-9 score of at least 10. Considering the difference in sampling rates between the daily sensor data and the weekly self-rating scales, a period-level label filling approach for each episode was adopted. This approach involved assigning a minimum of 14 consecutive positive labels (‘1’) to every day within a depressive episodic period.

A customized data quality control scheme was implemented to impute missing sensor data. To prevent spurious data non-stationarities from affecting the analytical outcomes, customized variable-specific selective mean imputation was performed. This algorithm fills the missing data instances in each variable (e.g., steps, total sleep duration) for each participant with the mean of that variable according to its associated label: missing data during euthymia is imputed with the variable-specific euthymic mean value, and similarly missing data during episodes with symptoms is imputed with the variable-specific symptomatic mean value.

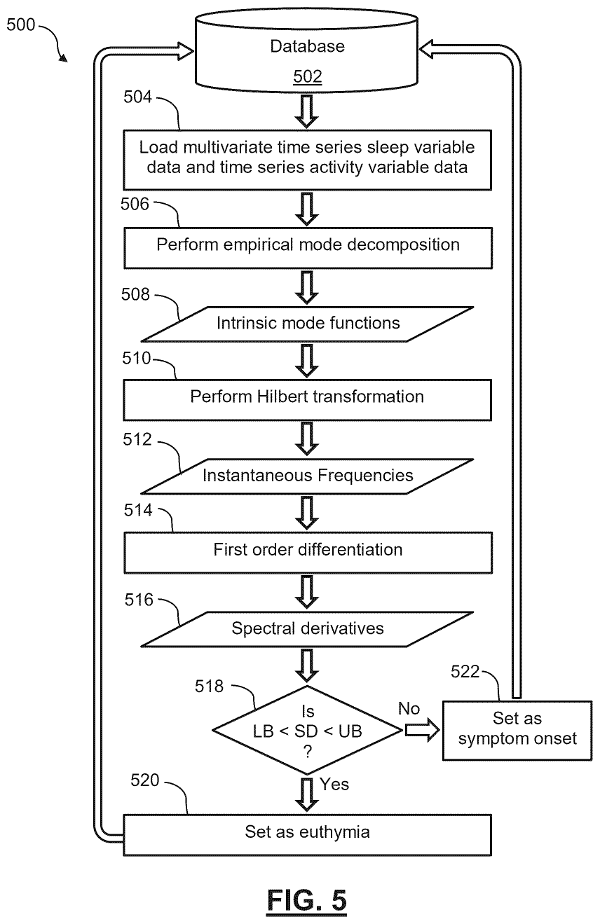

FIG. 5 is a flowchart showing the subsequent analytical pipeline 500 for the experimental evaluation. The experimental evaluation used the spectral derivatives, rather than the instantaneous frequencies, for spike detection.

A database 502 stores the actimetric patient data. At block 504, the multivariate time series sleep variable data and time series activity variable data are loaded and at block 506, empirical mode decomposition is performed to obtain the intrinsic mode functions at block 508. Hilbert transformation is performed at block 510 to obtain the instantaneous frequencies at block 512, and first order differentiation is performed at block 514 to obtain the spectral derivative at block 516. At block 518, the spectral derivative is compared to the upper and lower bounds. If the spectral derivative is between the upper and lower bounds (“yes” at step 518), at block 520 a flag is set to indicate euthymia and recorded in the database 502. Conversely, if the spectral derivative is outside of the upper and lower bounds (“no” at step 518), at block 522 a flag is set to indicate probable symptom onset and recorded in the database 502.

### Episode Onset Detection Performance Analysis

The sleep and activity data indicative of euthymia and depressive episodes were then compared with the target labels extracted from the self-rating scale data for episode onset detection performance analysis. As explained above, a total PHQ-9 score greater or equal to 10 for at least two consecutive weeks was labeled as a depressive episode. The performance of the method according to the present disclosure was evaluated using the weighted average of the following participant-specific metrics: accuracy (37), sensitivity (38), and specificity (39).

$$Accuracy = \frac{TP + TN}{TP + FP + TN + FN} \qquad ( 37 )$$

$$Sensitivity = \frac{TP}{TP + FN} \qquad ( 38 )$$

$$Specificity = \frac{TN}{TN + FP} \qquad ( 39 )$$

such that TP is the number of detected spikes up to 2 weeks prior to a self-reported episode, FP is the number of detected spikes in a period of self-reported euthymia, TN is the number of euthymic observations within self-reported euthymia, and FN is the number of euthymic observations within self-reported episodes.

### Depressive Episode Onset Detection Results

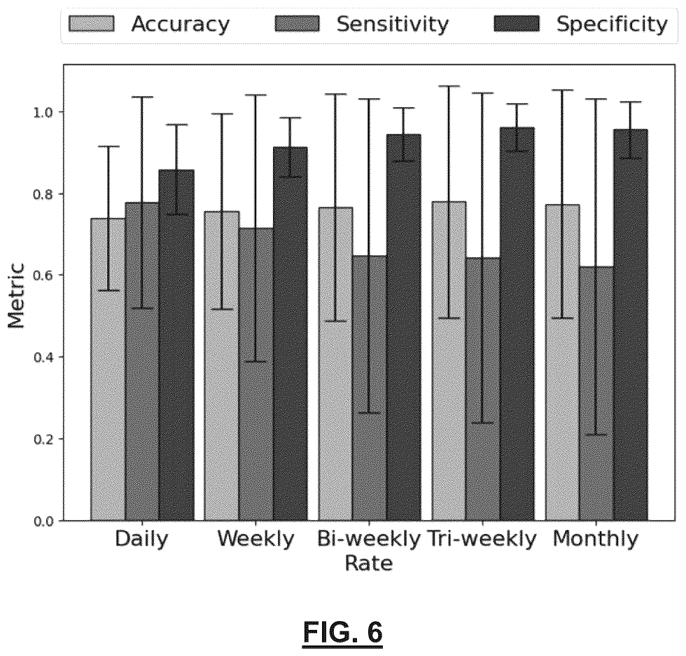

The experiment was intended to evaluate the onset detection performance of each separate variable at each separate time scale (i.e. each intrinsic mode function). For that reason, the experimental comparison did not include multiple comparisons and multivariate predictions. Using the daily step variable to represent time series activity variable data, the implementation of the method according to the present disclosure showed decreasing levels of episode onset detection sensitivity with decreasing time resolution. Sensitivity decreased from 0.78±0.26 for day-to-day patterns to 0.62±0.38 for monthly patterns. Conversely, the ability of the method to detect euthymic phases (i.e. specificity) increased with decreasing temporal resolution, starting at 0.86±0.11 using day-to-day patterns, reaching 0.96±0.07 for monthly patterns. The averaged performance metrics of the method across study participants are presented in FIG. 6 for time series activity variable data.

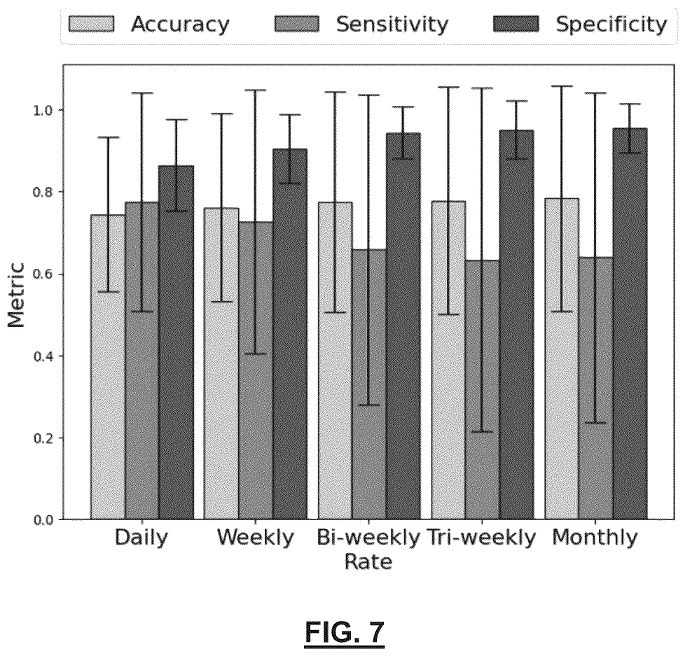

Similarly, using the daily total sleep variable to represent time series sleep variable data, a day-to-day sensitivity of 0.77±0.27 was achieved, decreasing to 0.64±0.40 for monthly total sleep patterns. As for specificity, the method showed a monotonic increase in euthymic phase detectability with the decrease of temporal resolution, from 0.86±0.11 for day-to-day total sleep patterns to 0.96±0.06 for monthly patterns. The averaged performance metrics of the method across study participants are presented in FIG. 7 for the time series sleep variable data.

While the above description outlines experimental results for using the method described herein to predict depressive episodes, a similar analysis has been conducted for prediction of (hypo)manic episodes, and the method described herein has also been found to have utility for prediction of (hypo)manic episodes.

Methods according to the present disclosure enable accurate symptom onset detection for a psychiatric illness or medical condition using passively collected activity data and sleep data. The method was evaluated on a real-world dataset to detect the onset of depressive episodes in bipolar disorder, and achieved high performance at different temporal resolutions.

The results demonstrate the impact of temporal resolution on the detection of symptom onset (e.g. depressive episode) and euthymic phases. Moreover, the decrease in sensitivity with decreasing time resolution suggests that more granular data collection provides more accurate identification of symptom onset. Conversely, the method's ability to detect euthymic phases with decreased temporal resolution has significant clinical implications. The increased specificity in identifying euthymic phases at coarser time intervals allows for a broader overview of stable mood periods, which is important for monitoring and managing psychiatric illness or medical condition.

### Technical Effect

As can be seen from the above description, the technology described herein represents significantly more than merely using categories to organize, store and transmit information and organizing information through mathematical correlations. The technology is in fact an improvement to the technology of monitoring previously diagnosed psychiatric illness or medical condition. The technology described herein provides for objective actimetric assessment of whether a user is likely to experience a symptom of that psychiatric illness or medical condition, and for notification where a symptom is predicted. This facilitates the ability of the user to prepare for the symptom and/or the ability of relevant personnel to respond. As such, the technology is confined to psychiatric monitoring applications. Moreover, it is to be appreciated that the present technology is not directed to methods of medical treatment or even to methods of diagnosing a particular disorder; it is applied, inter alia, where a diagnosis of a psychiatric illness or medical condition has already been made by a human medical practitioner. The technology provides an objective technique for detection of likely symptom onset within the context of an existing diagnosis, eliminating subjectivity by either doctor or patient. The present technology provides a diagnostic tool to replace subjective perception with objective measurement. In this sense, the present technology, while innovative in its application and implementation, is analogous in its result to a heart monitor for a patient already diagnosed with a cardiac condition, where the heart monitor uses sensor data to predict impending cardiac events (e.g., arrhythmias) so that intervention can be initiated. Likewise, the ability of the present technology to use actimetric data to predict symptom onset for a psychiatric illness or medical condition can enable timely intervention and facilitate appropriate treatment adjustments, ultimately resulting in improved patient outcomes. Furthermore, the reliable detection of symptom onset may offer valuable insights into the progression of illness or medical condition over time, allowing for the tracking of individual patterns and supporting personalized care. By providing clinicians with precise information about episode onset, the present technology enables them to tailor interventions and treatment plans according to the unique needs of each patient. Again, the present technology is a diagnostic tool which supports the provision of medical care by qualified human personnel, and no methods of medical treatment are claimed.

### Technical Environment and Embodiment

The present technology may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology have been described above with reference to flowchart illustrations and/or block diagrams of methods, apparatus (systems) and computer program products according to various embodiments. In this regard, the flowchart and block diagrams in the Figures illustrate the architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing may have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, other programmable data processing apparatus, or other devices to function in a particular manner, such that the instructions stored in the computer readable storage medium produce an article of manufacture including instructions which implement aspects of the functions/acts specified in the flowchart and/or block diagram block or blocks. The computer readable program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

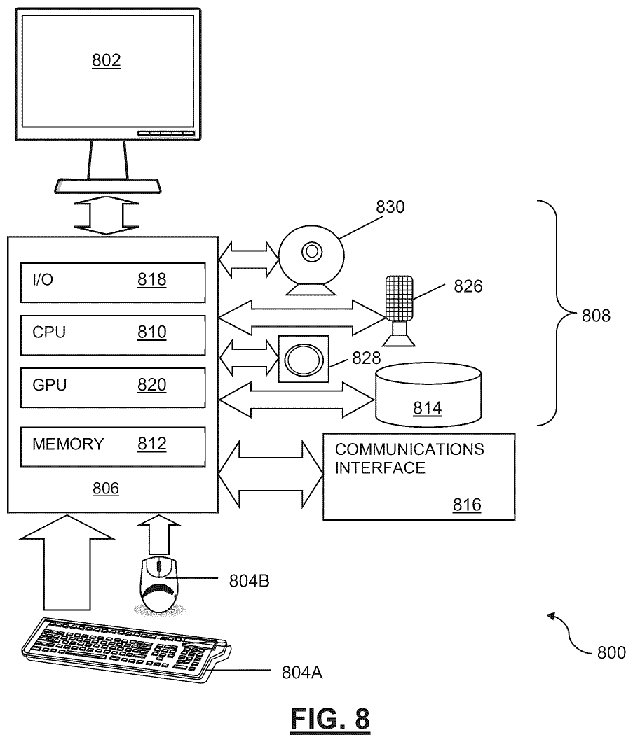

An illustrative computer system in respect of which the technology herein described may be implemented is presented as a block diagram in FIG. 8. The illustrative computer system is denoted generally by reference numeral 800 and includes a display 802, input devices in the form of keyboard 804A and pointing device 804B, computer 806 and external devices 808. While pointing device 804B is depicted as a mouse, it will be appreciated that other types of pointing device, or a touch screen, may also be used.

The computer 806 may contain one or more processors or microprocessors, such as a central processing unit (CPU) 810. The CPU 810 performs arithmetic calculations and control functions to execute software stored in an internal memory 812, preferably random access memory (RAM) and/or read only memory (ROM), and possibly additional memory 814. The additional memory 814 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 814 may be physically internal to the computer 806, or external as shown in FIG. 8, or both.

The computer system 800 may also include other similar means for allowing computer programs or other instructions to be loaded. Such means can include, for example, a communications interface 816 which allows software and data to be transferred between the computer system 800 and external systems and networks. Examples of communications interface 816 can include a modem, a network interface such as an Ethernet card, a wireless communication interface, or a serial or parallel communications port. Software and data transferred via communications interface 816 are in the form of signals which can be electronic, acoustic, electromagnetic, optical or other signals capable of being received by communications interface 816. Multiple interfaces, of course, can be provided on a single computer system 800.

Input and output to and from the computer 806 is administered by the input/output (I/O) interface 818. This I/O interface 818 administers control of the display 802, keyboard 804A, external devices 808 and other such components of the computer system 800. The computer 806 also includes a graphical processing unit (GPU) 820. The latter may also be used for computational purposes as an adjunct to, or instead of, the (CPU) 810, for mathematical calculations.

The external devices 808 include a microphone 826, a speaker 828 and a camera 830. Although shown as external devices, they may alternatively be built in as part of the hardware of the computer system 800.

The various components of the computer system 800 are coupled to one another either directly or by coupling to suitable buses.

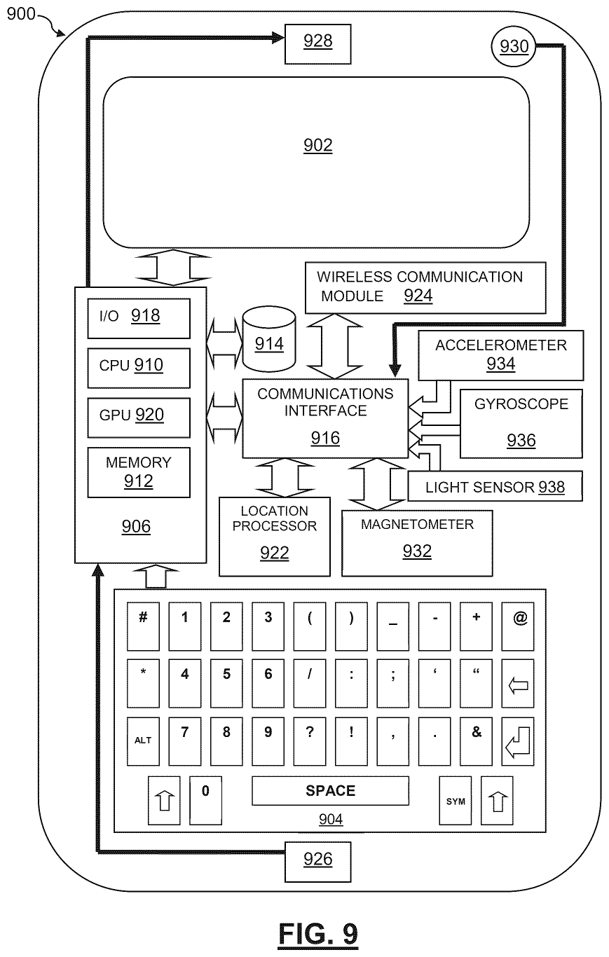

FIG. 9 shows an illustrative networked mobile wireless telecommunication computing device in the form of a smartphone 900. The smartphone 900 includes a display 902, an input device in the form of keyboard 904 and an onboard computer system 906. The display 902 may be a touchscreen display and thereby serve as an additional input device, or as an alternative to the keyboard 904. The onboard computer system 906 comprises a central processing unit (CPU) 910 having one or more processors or microprocessors for performing arithmetic calculations and control functions to execute software stored in an internal memory 912, preferably random access memory (RAM) and/or read only memory (ROM) is coupled to additional memory 914 which will typically comprise flash memory, which may be integrated into the smartphone 900 or may comprise a removable flash card, or both. The smartphone 900 also includes a communications interface 916 which allows software and data to be transferred between the smartphone 900 and external systems and networks. The communications interface 916 is coupled to one or more wireless communication modules 924, which will typically comprise a wireless radio for connecting to one or more of a cellular network, a wireless digital network or a Wi-Fi network. The communications interface 916 will also typically enable a wired connection of the smartphone 900 to an external computer system. A microphone 926 and speaker 928 are coupled to the onboard computer system 906 to support the telephone functions managed by the onboard computer system 906, and a location processor 922 (e.g. including GPS receiver hardware) may also be coupled to the communications interface 916 to support navigation operations by the onboard computer system 906. One or more cameras 930 (e.g. front-facing and/or rear facing cameras) may also be coupled to the onboard computer system 906, as may be one or more of a magnetometer 932, accelerometer 934, gyroscope 936 and light sensor 938. Input and output to and from the onboard computer system 906 is administered by the input/output (I/O) interface 918, which administers control of the display 902, keyboard 904, microphone 926, speaker 928, camera 930, magnetometer 932, accelerometer 934, gyroscope 936 and light sensor 938. The onboard computer system 906 may also include a separate graphical processing unit (GPU) 920. The various components are coupled to one another either directly or by coupling to suitable buses.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 912 of the onboard computer system 906 of the smartphone 900 or the memory 812 of the computer 806, or on a computer usable or computer readable medium external to the onboard computer system 906 of the smartphone 900 or the computer 806, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

### REFERENCES

The following list of references is provided for convenience only, without admission that any of the references are relevant to the present disclosure and without admission that any of the references are citable as prior art.
[1] M. Kang and K. Chai, “Wearable Sensing Systems for Monitoring Mental Health,” Sensors (Basel), vol. 22, no. 3, p. 994, January 2022. [Online]. Available: https://doi.org/10.3390/s22030994.
[2] M. Moitra et al., “Estimating the risk of suicide associated with mental disorders: A systematic review and meta-regression analysis,” J. Psychiatr. Res., vol. 137, pp. 242-249, 2021. [Online]. Available: https://doi.org/10.1016/j.jpsychires.2021.02.053.
[3] R. Garriga et al., “Machine learning model to predict mental health crises from electronic health records,” Nat. Med., vol. 28, pp. 1240-1248, 2022. [Online]. Available: https://doi.org/10.1038/s41591-022-01811-5.
[4] A. Abd-Alrazaq et al., “Systematic review and meta-analysis of performance of wearable artificial intelligence in detecting and predicting depression,” npj Digit. Med., vol. 6, p. 84, 2023. [Online]. Available: https://doi.org/10.1038/s41746-023-00828-5.
[5] A. Pratap et al., “Real-world behavioral dataset from two fully remote smartphone-based randomized clinical trials for depression,” Sci. Data, vol. 9, p. 522, 2022. [Online]. Available: https://doi.org/10.1038/s41597-022-01633-7.
[6] A. Klein et al., “Remote Digital Psychiatry for Mobile Mental Health Assessment and Therapy: MindLogger Platform Development Study,” J. Med. Internet Res., vol. 23, no. 11, p. e22369, November 2021. [Online]. Available: https://doi.org/10.2196/22369.
[7] S. B. Goldberg et al., “Mobile phone-based interventions for mental health: A systematic meta-review of 14 meta-analyses of randomized controlled trials,” PLOS Digital Health, vol. 1, no. 1, p. e0000002, 2022. [Online]. Available: https://doi.org/10.1371/journal.pdig.0000002.
[8] N. Gomes et al., “A Survey on Wearable Sensors for Mental Health Monitoring,” Sensors (Basel), vol. 23, no. 3, p. 1330 January 2023. [Online]. Available: https://doi.org/10.3390/s23031330.
[9] A. Ahmed et al., “Wearable devices for anxiety & depression: A scoping review,” Computer Methods and Programs in Biomedicine Update, vol. 3, pp. 100095, 2023. [Online]. Available: https://doi.org/10.1016/j.cmpbup.2023.100095.
[10] A. Ortiz et al., “Identifying patient-specific behaviors to understand illness or medical condition trajectories and predict relapses in bipolar disorder using passive sensing and deep anomaly detection: protocol for a contactless cohort study,” BMC Psychiatry, vol. 22, p. 288, 2022. [Online]. Available: https://doi.org/10.1186/s12888-022-03923-1.
[11] A. Ortiz et al., “Apps and gaps in bipolar disorder: A systematic review on electronic monitoring for episode prediction,” J. Affect. Disord., vol. 295, pp. 1190-120 December 2021. [Online]. Available: https://doi.org/10.1016/j.jad.2021.08.140.
[12] R. Syed et al., “Digital Health Data Quality Issues: Systematic Review,” J. Med. Internet Res., vol. 25, p. e42615, 2023. [Online]. Available: https://doi.org/10.2196/42615.
[13] A. Ortiz et al., “Nonlinear dynamics of mood regulation in bipolar disorder,” Bipolar Disord., vol. 17, no. 2, pp. 139-149, March 2015. [Online]. Available: https://doi.org/10.1111/bdi.12246.
[14] A. Sadilek et al., “Modeling Fine-Grained Dynamics of Mood at Scale,” in Proceedings of the ACM International Conference on Web Search and Data Mining (WSDM), Rome, Italy, 2013, pp. 3-6.
[15] A. Ortiz et al., “The futility of long-term predictions in bipolar disorder: mood fluctuations are the result of deterministic chaotic processes,” Int. J. Bipolar Disord., vol. 9, p. 30, 2021. [Online]. Available: https://doi.org/10.1186/s40345-021-00235-3.
[16] R. D'Mello et al., “Similarity matrix-based anomaly detection for clinical intervention,” Sci. Rep., vol. 12, p. 9162, 2022. [Online]. Available: https://doi.org/10.1038/s41598-022-12792-3.
[17] T. Saito, H. Suzuki, and A. Kishi, “Predictive Modeling of Mental Illness or medical condition Onset Using Wearable Devices and Medical Examination Data: Machine Learning Approach,” Front. Digital Health, vol. 4, 2022. [Online]. Available: https://doi.org/10.3389/fdgth.2022.861808.
[18] B. Löwe et al., “Monitoring depression treatment outcomes with the patient health questionnaire-9,” Med. Care, vol. 42, no. 12, pp. 1194-121 December 2004. [Online]. Available: https://doi.org/10.1097/00005650-200412000-00006.
[19] Peter, D., Welch. (1967). “The use of fast Fourier transform for the estimation of power spectra: A method based on time averaging over short, modified periodograms”. IEEE Transactions on Audio and Electroacoustics, 15(2): 70-73. doi: 10.1109/TAU.1967.1161901.
[20] I. Daubechies, “The wavelet transform, time-frequency localization and signal analysis,” in IEEE Transactions on Information Theory, vol. 36, no. 5, pp. 961-15 Sep. 1990, doi: 10.1109/18.57199.
[21] Flandrin, P., G. Rilling, and P. Goncalves. 2004. “Empirical Mode Decomposition as a Filter Bank.” IEEE Signal Processing Letters 11(2): 112-14.
[22] Torres, María E., Marcelo A. Colominas, Gastón Schlotthauer, and Patrick Flandrin. 2011. “A Complete Ensemble Empirical Mode Decomposition with Adaptive Noise.” In 2011 IEEE International Conference on Acoustics, Speech and Signal Processing (ICASSP), 4144-47.
[23] Altman et al; BIOL PSYCHIATRY 1997; 42:948-955.

Certain currently preferred embodiments have been described by way of example. It will be apparent to persons skilled in the art that a number of variations and modifications can be made without departing from the scope of the claims. In construing the claims, it is to be understood that the use of a computer to implement the embodiments described herein is essential.

What is claimed is:

1. A sensor-based method for individualized prediction of onset of an indication of a psychiatric medical condition, the method comprising:
obtaining actimetric patient data from at least one actimetric sensor associated with a patient, wherein the actimetric patient data comprises at least one of time series sleep variable data and time series activity variable data;
applying multi-resolution time-frequency analysis to the actimetric patient data to obtain time-frequency spike train data for the patient;
predicting onset of the indication from the time-frequency spike train data; and
issuing an alert when the onset of the indication is predicted from the time-frequency spike train data.

2. The method of claim 1, wherein applying multi-resolution time-frequency analysis to the actimetric patient data to obtain time-frequency spike train data for the patient comprises:
decomposing the actimetric patient data using empirical mode decomposition to obtain a plurality of temporal modes each representing a respective temporal scale, wherein each temporal mode is represented by an intrinsic mode function.

3. The method of claim 2, wherein applying multi-resolution time-frequency analysis to the actimetric patient data to obtain time-frequency spike train data for the patient further comprises:
after decomposing the actimetric patient data, transforming the intrinsic mode functions to obtain one respective time series of instantaneous frequencies for each one of the intrinsic mode functions.

4. The method of claim 3, wherein transforming the intrinsic mode functions to obtain the respective time series of instantaneous frequencies of the intrinsic mode functions uses Hilbert transformation.

5. The method of claim 3, wherein applying multi-resolution time-frequency analysis to the actimetric patient data to obtain time-frequency spike train data for the patient comprises performing spike detection on the instantaneous frequencies.

6. The method of claim 5, wherein performing spike detection on the instantaneous frequencies comprises:
identifying instantaneous frequencies whose magnitudes exceed respective predetermined limits as spikes representing time-frequency anomalies associated with changes in variability within the actimetric patient data.

7. The method of claim 3, wherein applying multi-resolution time-frequency analysis to the actimetric patient data to obtain time-frequency spike train data for the patient further comprises:
after transforming the intrinsic mode functions to obtain the respective time series of instantaneous frequencies of the intrinsic mode functions, taking a first order derivative of each of the time series of instantaneous frequencies to obtain a spectral derivative time series for each of the intrinsic mode functions.

8. The method of claim 7, wherein applying multi-resolution time-frequency analysis to the actimetric patient data to obtain time-frequency spike train data for the patient further comprises performing spike detection on the spectral derivative time series.

9. The method of claim 8, wherein performing spike detection on the spectral derivative time series comprises:
identifying spectral derivatives whose magnitudes exceed respective predetermined limits as spikes representing time-frequency anomalies associated with changes in variability within the actimetric patient data.

10. The method of claim 9, wherein predicting onset of the indication from the time-frequency spike train data comprises computing anomaly scores for the time-frequency spike train data.

11. The method of claim 10, wherein predicting onset of the indication from the time-frequency spike train data further comprises comparing the anomaly scores to a threshold.

12. The method of claim 1, wherein the at least one actimetric sensor includes at least one body-worn sensor.

13. The method of claim 1, wherein the actimetric patient data comprises both time series sleep variable data and time series activity variable data.

14. The method of claim 1, wherein the indication is a depressive episode.

15. The method of claim 14, wherein the psychiatric medical condition is bipolar disorder.

16. The method of claim 14, wherein the psychiatric medical condition is major depressive disorder.

17. The method of claim 1, wherein the indication is one of a manic episode and a hypomanic episode.

18. One or more non-transitory computer-readable medium comprising instructions that, when executed by at least one processor of a data processing system, cause the data processing system to:
obtain actimetric patient data from at least one actimetric sensor associated with a patient, wherein the actimetric patient data comprises at least one of time series sleep variable data and time series activity variable data;
apply multi-resolution time-frequency analysis to the actimetric patient data to obtain time-frequency spike train data for the patient;
predict onset of an indication of a psychiatric medical condition from the time-frequency spike train data; and
issue an alert when the onset of the indication is predicted from the time-frequency spike train data.

19. A system for individualized prediction of onset of an indication of a psychiatric medical condition, the system comprising:
at least one actimetric sensor, wherein the least one actimetric sensor is configured to obtain actimetric patient data comprising at least one of time series sleep variable data and time series activity variable data;
at least one data processing system communicatively coupled to the at least one actimetric sensor, wherein the at least one data processing system comprises at least one processor and memory coupled to the at least one processor, wherein the memory contains instructions which, when implemented by the at least one processor, cause the at least one processor to:
obtain actimetric patient data from the at least one actimetric sensor associated with a patient, wherein the actimetric patient data comprises at least one of time series sleep variable data and time series activity variable data;
apply multi-resolution time-frequency analysis to the actimetric patient data to obtain time-frequency spike train data for the patient;
predict onset of the indication from the time-frequency spike train data; and
issue an alert when the onset of the indication is predicted from the time-frequency spike train data.

20. The system of claim 19, wherein the at least one actimetric sensor includes at least one body-worn sensor.
